(19) United States
(12) Patent Application Publication
(10) Pub. No.: US 2005/0203467 A1
(43) Pub. Date: Sep. 15, 2005

(54) Trocar seal

(76) Inventors: Peter T. O'Heeron, Houston, TX (US); Ivan W. Byford, Houston, TX (US)

Correspondence Address: Clarence E. Eriksen;Jackson Walker L.L.P., Suite 2100, 112 E. Pecan Street, San Antonio, TX, 78205-1521, US

(21) Appl. No.: 10/800,783

(22) Filed: Mar. 15, 2004

(51) Int. Cl. A61M 5/00

(52) U.S. Cl. 604/249; 604/246; 604/167.01

(57) ABSTRACT

A seal is disclosed for installation on the proximal end of a trocar. The seal is generally cylindrical in shape and has an upper portion and a lower portion. The lower portion is formed for mating engagement with the proximal end of the trocar. The upper portion comprises a central hole for insertion of a surgical instrument and a plurality of folds in proximity to the central hole. The folds allow the central hole to dilate and contract to seal on surgical instruments of varying diameters, and various configurations of folds are disclosed.

### BACKGROUND OF THE INVENTION

1. Field of the Invention

The present invention relates to surgical instruments known as trocars which are used in endoscopic surgery to pierce or puncture an anatomical cavity to provide communication with the cavity during a surgical procedure. More particularly, the present invention relates to a seal to prevent the escape of insufflated gas during the performance of surgical procedures using the trocar.

2. Description of the Prior Art

Endoscopic surgery is an essential method of performing surgical operations and has become the surgical procedure of choice, because of its patient care advantages over “open surgery.” One form of endoscopic surgery is laparoscopic surgery, and a significant advantage of laparoscopic surgery over open surgery is the decreased post-operative recovery time. In most instances, a patient is able to leave the hospital within hours after laparoscopic surgery has been performed. With open surgery, several days of hospital care may be required before the patient is discharged. Additionally, laparoscopic surgery achieves decreased incidents of post-operative abdominal adhesions, decreased post-operative pain, and enhanced cosmetic results.

Conventionally, a laparoscopic surgical procedure begins with the insufflation of the abdominal cavity with carbon dioxide. The introduction of this gas into the abdominal cavity lifts the abdominal wall away from the internal viscera. The abdominal wall is then penetrated with a device known as a trocar, which includes a housing assembly, a cannula assembly attached to the housing assembly to form a bore through the trocar, and a piercing element called an obturator. The obturator slides through an access port formed on the upper (i.e. proximal) end of the housing assembly and through the bore of the trocar. The obturator has a diameter which is substantially the same as the diameter of the access port. After insertion of the trocar through the abdominal wall of the patient, the obturator is removed by the surgeon while leaving the cannula or tube protruding through the body wall. Laparoscopic instruments can then be inserted through the cannula to view internal organs and to perform surgical procedures.

Once the obturator is removed from the bore of the housing, it is necessary to obstruct the access port so that the carbon dioxide gas introduced into the abdominal cavity of the patient is contained. Traditionally, a trocar includes a spring-loaded flapper valve which opens when the obturator is inserted and which closes when the obturator is removed from the cannula to keep the insufflated gas from escaping. However, the insertion of laparoscopic instruments into the trocar re-opens the flapper door. To prevent escape of the insufflated gas upon insertion of a laparoscopic instrument, a trocar also comprises a seal which is capable of providing sealing for laparoscopic instruments having varying diameters e.g. between 5 mm and 12 mm. Since such seals are capable of providing sealing during the same surgical procedure for laparoscopic instruments of varying diameters, they are commonly referred to as “universal” seals.

Various designs of universal seals have been proposed. See for example, U.S. Pat. Nos. 5,350,364; 5,385,553; 5,407,433; 5,512,053; 5,628,732, 5,827,228; 5,342,315; and 4,112,932. Such prior art seals comprise a plurality of mechanical parts which must be assembled and are usually expensive.

### SUMMARY OF THE INVENTION

In accordance with the present invention, a seal is provided for installation on a trocar having a proximal end with an access port at the proximal end. The seal is generally cylindrical in shape and has an upper portion and a lower portion, with the lower portion of the seal being formed for mating engagement with the access port at the proximal end of the trocar. The upper portion of the seal has a central hole formed therein to permit a surgical instrument to pass through the seal. A plurality of folds are formed the upper portion of the seal in proximity to the central hole. The folds in the upper portion of the seal comprise stored seal material which allows the central hole in the seal to dilate or contract to seal on a surgical instrument inserted through the central hole, and which allows for lateral and vertical movement of a surgical instrument without loss of sealing function.

The central hole may for example be approximately 2.0 millimeters in diameter. Preferably the folds that are in proximity to the central hole in the seal allow the central hole opening in the seal to be expendable to about 13 millimeters. Thus, a seal in accordance with the present invention is capable of providing the sealing function for surgical instruments of varying diameters between approximately 2.0 millimeters and 13 millimeters.

A seal in accordance with the present invention may be fabricated from any suitable pliable material using a molding process, and is preferably fabricated from silicone or other approved pliable rubber or plastic.

In one embodiment, a seal in accordance with the present invention comprises a plurality of accordion-like folds which radiate outwardly from the central hole. The number of accordion-like folds that the seal contains will determine the maximum diameter to which the central hole in the seal can be expanded. Preferably, a seal in accordance with the present invention has between 2 and 75 accordion-like folds.

In another embodiment, a seal in accordance with the present invention comprises a plurality of circular folds in the upper portion which are concentric with the central hole. The number of circular folds will determine the range of diameter of surgical instruments that the seal can accommodate without loss of the sealing function. In a third embodiment, a seal in accordance with the present invention comprises a plurality of folds in the upper portion which are spiral in shape, while yet a fourth embodiment comprises folds in the upper portion of the seal which are petal-shaped. Finally, a seal in accordance with the present invention may comprise folds in the upper portion which are indent folds.

All embodiments of a seal in accordance with the present invention may be formed with a recess in the upper portion with the central hole being located at the bottom of the recess. The recess enhances the ability of the surgeon to make a one-handed insertion of a surgical instrument, into the trocar, instead of steadying the instrument with one hand and inserting the instrument it with the other hand.

A trocar having any of the foregoing described seals constitutes an improvement over the prior art.

### BRIEF DESCRIPTION OF THE DRAWINGS

FIG. 1 is an exploded perspective view of an exemplary embodiment of a trocar.

FIG. 2 is a lateral section view of the proximal end of the body of the trocar shown in FIG. 1.

FIG. 3A is a perspective view of one embodiment of a trocar seal in accordance with the present invention.

FIG. 3B is a perspective view of a the embodiment of the trocar seal FIG. 3A with a recess in the upper portion of the seal.

FIG. 4 is an enlarged perspective view of the accordion-like folds 44 in FIGS. 3A and 3B.

FIGS. 5A and 5B are perspective views of alternative configurations of the embodiments of the trocar seals shown in FIGS. 3A and 3B, respectively.

FIG. 6 is a lateral section view of a proximal end of the trocar illustrated in FIG. 1 with a seal in accordance with the present invention installed.

FIG. 7 is a perspective view of another embodiment of a trocar seal in accordance with the present invention.

FIG. 8 is a perspective view of another embodiment of a trocar seal in accordance wit the present invention.

FIG. 9 is a perspective view of another embodiment of a trocar seal in accordance with the present invention.

FIG. 10 is a perspective view of another embodiment of a trocar seal in accordance with the present invention.

### DESCRIPTION OF THE PREFERRED EMBODIMENTS

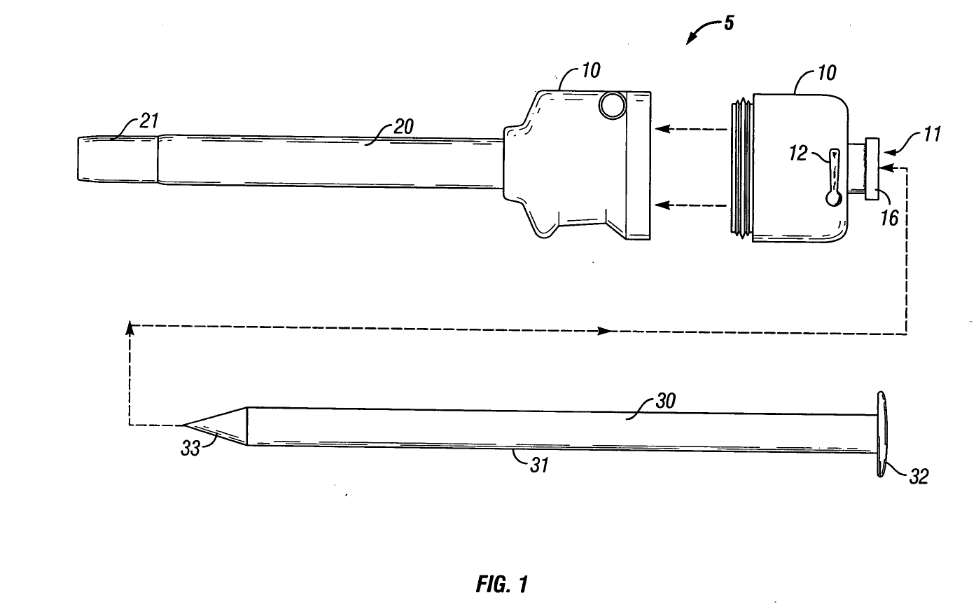

With reference to FIG. 1, an embodiment of a trocar 5 comprises a housing assembly 10 to which is attached a cannula assembly 20. The cannula assembly 20 is a hollow tube, and when attached to the housing assembly 10, a bore is defined through the trocar 5. An access port 11 is formed in the proximal end of the housing assembly 10 and includes flange 16. The access port 11 and the bore defined by the cannula assembly 20 are axially aligned. The diameter of the access port 11 may, for example, be between 2 mm and 22 mm.

Still with reference to FIG. 1, a trocar 5 also includes an obturator assembly 30 having a shaft 31 with an arcuate-shaped cap 32 at the proximal end of the shaft and a piercing tip 33 at the distal end of the shaft. The obturator assembly 30 has a diameter substantially the same as the diameter of the access port 11, and the obturator 30 is inserted to the housing assembly 10 through the access port 11. The obturator slides in the bore that is defined by the combination of housing assembly 10 and cannula assembly 20. The trocar 5 may comprise a safety shield 21, although the present invention is not limited to seals for trocars with safety shields.

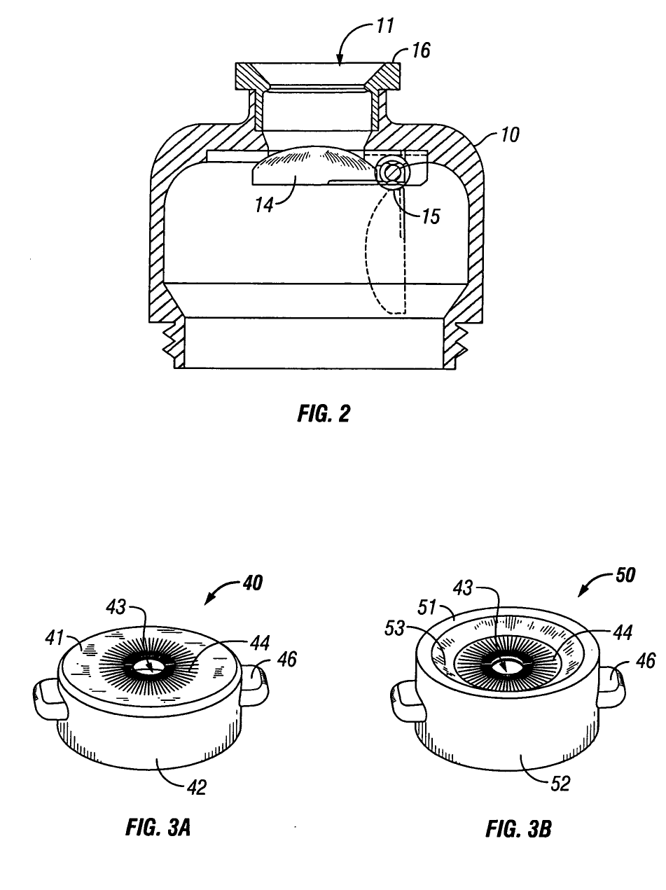

With reference to FIG. 2, a trocar 5 includes a flapper valve door 14 for regulating communication through the access port 11. As illustrated in FIG. 2, the flapper valve door 14 may have a domed protrusion 14A which engages the housing 10 to form a seal when the flapper valve door 14 is closed. The flapper valve door 14 is rotatably connected to the housing assembly 10, and the flapper valve door 14 is rotatable between a closed position and an open position, as shown by the dotted lines in FIG. 2. Resistance mechanisms, such as torsion springs or compression springs (not shown) may be used to bias the flapper valve door 14 in the closed position. A manual flapper door actuator 12 (FIG. 1) is provided for manual rotation of the flapper valve door 14 between the closed position and the range of open positions. Withdrawal of the obturator assembly 30 from the housing assembly 10 results in the closure of the flapper valve door.

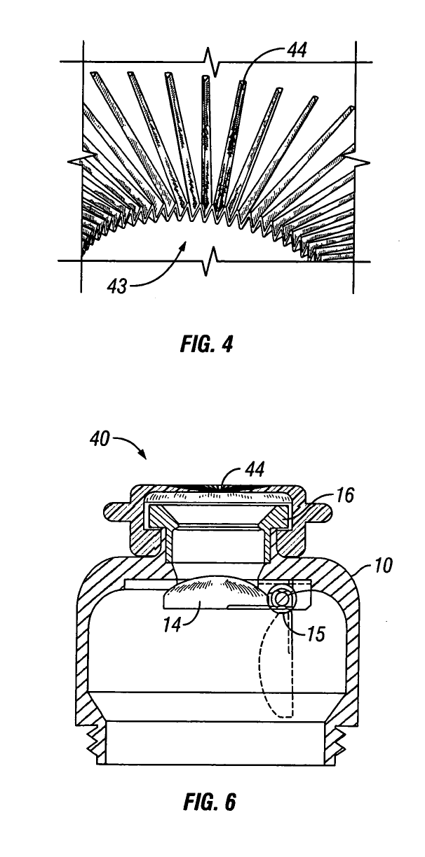

After the obturator assembly 30 is withdrawn from the trocar 5, a seal in accordance with the present invention is attached to access port 11, and one embodiment of such a seal is illustrated in FIG. 3A. Seal 40 is generally cylindrical in shape with an upper portion 41 and a lower portion 42. The lower portion 42 is formed as shown in FIG. 6 for mating engagement with the flange 16 at the access port 11 at the proximal end of the trocar and seal 40 may have ears 46 to facilitate the disengagement of seal 40 from the proximal end of the trocar. The upper portion 41 of seal 40 has a central hole 43 formed therein to permit a surgical instrument to pass through the seal. A plurality of accordion-like folds 44 are formed in the upper portion 41 in a generally circular pattern around the central hole 43. A portion of those accordion-like folds 44 is illustrated in FIG. 4. Seal 40 is formed from a suitable pliable material using a molding process and silicone or other approved flexible rubber or plastic is the preferred material for the seal. The silicone material is also preferably coated with a paralene or other approved coating so that a surgical instrument will not drag on the seal as it is inserted or withdrawn.

With reference now to FIG. 3B, there is illustrated a second seal 50 in accordance with the present invention. Seal 50 is also generally cylindrical in shape with an upper portion 51 and a lower portion 52. The lower portion 52 of seal 50 is also formed like the lower portion of seal 40 for mating engagement with the access port 11 of trocar 5 (see FIG. 6). Seal 50 differs from seal 40 in that the upper portion 51 of seal 50 has a recess 53 formed therein and the central hole 43 and the plurality of accordion-like folds 44 are formed at the bottom of the recess 53. The accordion-like folds again are in a generally circular pattern around central hole 43. The recess 53 is provided to enhance the ability of the surgeon to make a one-handed insertion of surgical instruments through the seal.

The number of accordion-like folds 44 surrounding the central hole 43 in either seal 40 or seal 50 will determine the extent to which central hole 43 is expandable to accommodate surgical instruments of varying diameters, and a seal in accordance with the present invention will have between two and seventy-five folds. It is believed that the use of 75 accordion-type folds 44 will permit the central hole 43 to be expandable from 2.0 mm to 13 mm while still maintaining the sealing function with the surgical instruments.

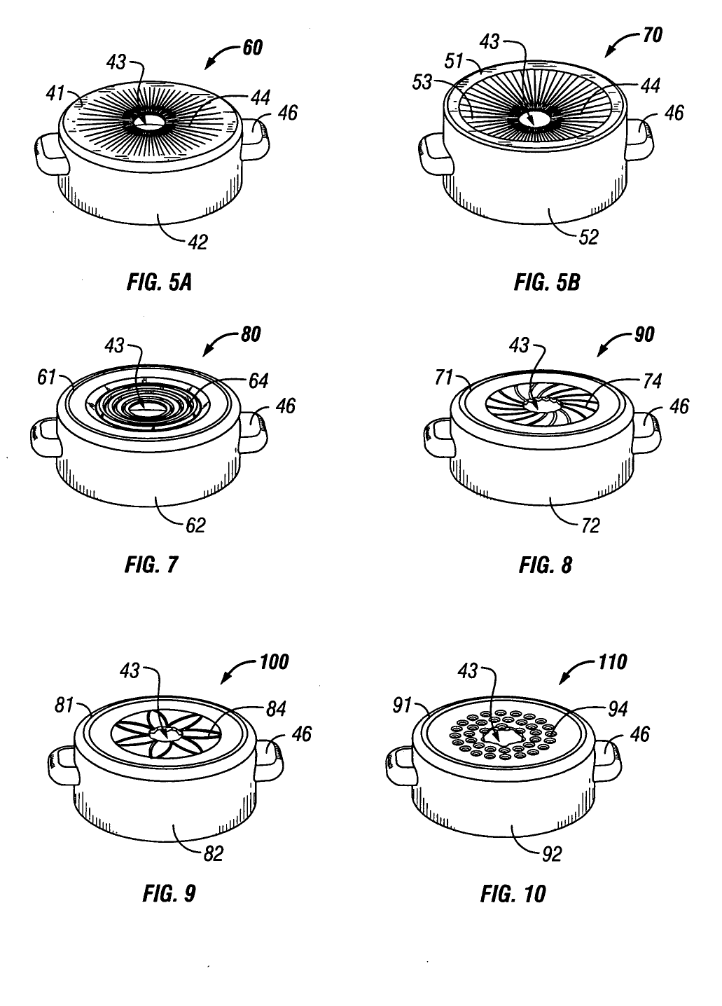

With reference now to FIGS. 5A and 5B, alternative embodiments of the seals of FIGS. 3A and 3B, respectively, are illustrated. Seals 60 and 70 illustrated in FIGS. 5A and 5B differ from seals 40 and 50 of FIGS. 3A and 3B in that the accordion-like folds radiate further outward from the central hole 43 than do the folds in the seals shown in FIGS. 3A and 3B. It is believed that the radial length of the folds will affect the extent to which the seal will dilate to accommodate surgical instruments of varying diameters.

FIGS. 7-10 illustrate alternative embodiments of a seal in accordance with the present invention. In FIG. 7, seal 80 is illustrated which has an upper portion 61 in which a plurality of circular folds 64 are formed. The circular folds 64 are concentric with central hole 43. In FIG. 8, seal 90 has a plurality of spiral folds 74 formed in the upper portion 71. These spiral folds 74 curve outwardly from central hole 43. In FIG. 9, seal 100 has a plurality of petal-shaped seals 84 formed in the upper portion 81, and in FIG. 10, a plurality of indent folds 94 are formed in the upper portion 91 of seal 110.

The bottom portions 62, 72, 82 and 92 of the seals illustrated in FIGS. 7-10, respectively, are formed for mating engagement with the flange 16 at the access port 11, in the manner illustrated in FIG. 6.

Each of the seals illustrated in FIGS. 7-10 may have a recess formed therein in its upper portion as illustrated in FIGS. 3B and 5B to enhance the surgeon's ability to make a one-handed insertion of a surgical instrument through the seal. When a recess is used, the central hole 43 will be at the bottom of the recess and the fold pattern will be formed in proximity to the central hole along the wall of the recess.

It is believed that a seal fabricated in accordance with the present invention will have a number of advantages over prior art seals. For example, the folds tend to form a cuff around the inserted surgical instrument at the distal end of the seal which permits sealing to be maintained during lateral movement the surgical instrument. Further, during withdrawal of the surgical instrument and tissue from the trocar, the folds may reverse thereby making it easier to remove the surgical instrument and the tissue. Third, the folds allow fluids to channel back into the trocar. Fourth, the folds themselves will allow the surgeon to funnel surgical instruments into the trocar instead of steadying the instrument with one hand and inserting it with the other hand, and the ability to funnel surgical instruments into the trocar will be enhanced by forming a recess in the upper portion of the seal.

1. A seal for installation on a trocar having a proximal end that has an access port, which seal is generally cylindrical in shape and has upper portion and lower portion, the lower portion of the seal being formed for mating engagement with the access port at the proximal end of the trocar and the upper portion of the seal having: (a) a central hole formed therein to permit a surgical instrument to pass through the seal; and (b) a plurality of folds formed in the upper portion in proximity to the central hole.

2. The seal of claim 1, wherein the folds comprise a plurality of substantially straight accordion-like folds which radiate outwardly from the central hole.

3. The seal of claim 2, wherein the upper portion of the seal has a recess formed therein and the central hole is at the bottom of the recess.

4. The seal of claim 2, wherein the central hole in the seal is approximately 2.0 millimeters in diameter.

5. The seal of claim 4, wherein the central hole in the seal is expandable from approximately 2.0 millimeters to about thirteen millimeters.

6. The seal of claim 2, wherein it comprises a pliable material.

7. The seal of claim 6, wherein the pliable material is silicone.

8. The seal of claim 2, wherein the accordion-like folds in the seal number between two and seventy-five.

9. The seal of claim 1, wherein the folds comprise a plurality of circular folds, each of which is concentric with the central hole.

10. The seal of claim 9, wherein the upper portion of the seal has a recess formed therein and the central hole is at the bottom of the recess.

11. The seal of claim 9, wherein the central hole in the seal is approximately 2.0 millimeters in diameter.

12. The seal of claim 11, wherein the central hole in the seal is expandable from approximately 2.0 millimeters to about thirteen millimeters.

13. The seal of claim 9, wherein it comprises a pliable material.

14. The seal of claim 13, wherein the pliable material is silicone.

15. The seal of claim 1, wherein the folds comprise a plurality of spiral-like folds which radiate outwardly from the central hole.

16. The seal of claim 15, wherein the upper portion of the seal has a recess formed therein and the central hole is at the bottom of the recess.

17. The seal of claim 15, wherein the central hole in the seal is approximately 2.0 millimeters in diameter.

18. The seal of claim 17, wherein the central hole in the seal is expandable from approximately 2.0 millimeters to about thirteen millimeters.

19. The seal of claim 15, wherein it comprises a pliable material.

20. The seal of claim 19, wherein the pliable material is silicone.

21. The seal of claim 1, wherein the folds comprise a plurality of petal-shaped folds.

22. The seal of claim 21, wherein the upper portion of the seal has a recess formed therein and the central hole is at the bottom of the recess.

23. The seal of claim 21, wherein the central hole in the seal is approximately 2.0 millimeters in diameter.

24. The seal of claim 28, wherein the hole in the seal is expandable from approximately 2.0 millimeters to about thirteen millimeters.

25. The seal of claim 21, wherein it comprises a pliable material.

26. The seal of claim 25, wherein the pliable material is silicone.

27. The seal of claim 1, wherein the folds comprise a plurality of indent folds in proximity to the central hole.

28. The seal of claim 27, wherein the upper portion of the seal has a recess formed therein and the central hole is at the bottom of the recess.

29. The seal of claim 27, wherein the central hole in the seal is approximately 2.0 millimeters in diameter.

30. The seal of claim 29, wherein the central hole in the seal is expandable from approximately 2.0 millimeters to about thirteen millimeters.

31. The seal of claim 27, wherein it comprises a pliable material.

32. The seal of claim 31, wherein the pliable material is silicone.
